(19) United States
(12) Patent Application Publication
(10) Pub. No.: US 2025/0207151 A1
(43) Pub. Date: Jun. 26, 2025

(54) Population-Hastened Assembly Genetic Engineering

(71) Applicant: Massachusetts Institute of Technology, Cambridge, MA (US)

(72) Inventors: Joseph M. Jacobson, Newton, MA (US); Noah Jakimo, Boston, MA (US); Lisa Nip, Malden, MA (US)

(73) Assignee: Massachusetts Institute of Technology, Cambridge, MA (US)

(21) Appl. No.: 19/009,901

(22) Filed: Jan. 3, 2025

Related U.S. Application Data
(63) Continuation of application No. 16/443,841, filed on Jun. 17, 2019, now abandoned.
Application No. 16/443,841 is a continuation of application No. 15/045,243, filed on Feb. 16, 2016, now abandoned.
(60) Provisional application No. 62/116,543, filed on Feb. 15, 2015.

(51) Int. Cl. C12N 15/90; C12N 9/16

(52) U.S. Cl. CPC C12N 15/902; C12N 9/16; C07K 2319/80; C07K 2319/85

(57) ABSTRACT

Population-Hastened Assembly Genetic Engineering is a method for continuous genome recoding using a mixed population of cells. Nucleic acid donors are distributed amongst a population of cells that continuously transfer nucleic acids to achieve asynchronous recoding of genetic information within a subpopulation of the cells. Recombination is achieved with biochemical systems compatible with virtually any organism. An engineered directed endonuclease comprises a nucleic acid recognition domain, a nucleic acid endonuclease domain, and a linker fusing or causing interaction between the nucleic acid recognition domain and the nucleic acid endonuclease domain. The method includes causing at least one engineered directed endonuclease to create a nick in a nucleic acid strand, the nick being offset from the recognition sequence of the nucleic acid recognition domain; causing homologous recombination of the strand with a donor nucleotide to create a modified genome; and replicating the modified genome.

### RELATED APPLICATIONS

This application is a continuation of U.S. patent application Ser. No. 16/443,841, filed Jun. 17, 2019, which is a continuation of U.S. patent application Ser. No. 15/045,243, filed Feb. 16, 2016, which claims the benefit of U.S. Provisional Application Ser. No. 62/116,543, filed Feb. 15, 2015, the entire disclosures of which are herein incorporated by reference.

### FIELD OF THE TECHNOLOGY

The present invention relates to synthetic biology and, in particular, to methods for programmable modification of DNA.

### BACKGROUND

Genome recoding in a living organism is a highly multiplexed process that requires many donor nucleic acid sequences to template changes to precise positions on the genome. The process must then incorporate donor sequences into the correct position on the genome. In Multiplex Automated Genome Engineering (MAGE) [Gallagher R R, Li Z, Lewis A O, Isaacs F J. Rapid editing and evolution of bacterial genomes using libraries of synthetic DNA. Nat Protoc. 2014 October;9(10):2301-16], the mechanism of incorporation occurs when synthetic ssDNA oligonucleotides, assisted by lambda Red recombination, hybridize to the lagging strand of the DNA replication fork. Thus, said ssDNA would be analogous to Okazaki fragments, but containing mismatches that confer the desired mutation after surviving mismatch repair pathways before the next replication cycle.

Although the role of ssDNA in lambda Red recombination was known by 1997 [Hill S A, Stahl M M, Stahl F W. Single-strand DNA intermediates in phage λ's Red recombination pathway. Proceedings of the National Academy of Sciences of the United States of America 1997;94(7):2951-2956] and identified in 2010 [Mosberg J A, Lajoie M J, Church G M. Lambda red recombineering in Escherichia coli occurs through a fully single-stranded intermediate. Genetics. 2010 November;186(3):791-9] to be sufficient nucleic acid content for recombination in E coli, the application of MAGE to other organisms has been challenging. The technique has only been demonstrated in a few bacterial species as well as an engineered S. cerevisiae [DiCarlo J E, Conley A J, Penttilä M, Jäntti J, Wang H H, Church G M. Yeast oligo-mediated genome engineering (YOGE). ACS Synth Biol. 2013 Dec. 20;2(12):741-9]. Furthermore, the number of genomic positions in an individual cell that can be mutagenized via MAGE is limited by the number of ssDNA donors that can be transfected into the cell or internally expressed. This limitation is likely to prevent broad mutagenesis of the genome by either method of ssDNA introduction.

In Conjugative Assembly Genome Engineering (CAGE) [Gallagher R R, Li Z, Lewis A O, Isaacs F J. Rapid editing and evolution of bacterial genomes using libraries of synthetic DNA. Nat Protoc. 2014 October;9(10):2301-16], the mechanism of incorporation occurs when a donor bacterial cell mates with a recipient cell via an F pilus and delivers a copy of part of its genome, beginning from an origin of Transfer (oriT) sequence on the genome. The delivered DNA recombines with the recipient's genome and contains a marker element that enables selection of successful recombinants among the recipients. Incorporating all desired changes to the genome requires several rounds of pairing donor and recipients through a tournament-like bracket (binary heap) that assembles the genome in a hierarchical manner. The rigid structure of this process demands careful and laborious handling of materials.

Alternative recombinase-based approaches, such as Recombinase-Assisted Genome Assembly (RAGE) [Santos C N, Yoshikuni Y. Engineering complex biological systems in bacteria through recombinase-assisted genome engineering. Nat Protoc. 2014;9(6):1320-36] and methods used in the Synthetic Yeast 2.0 project [Annaluru N et al. Total synthesis of a functional designer eukaryotic chromosome. Science. 2014 Apr. 4;344(6179):55-8], are similarly limited in the range of positions in the genome that can be simultaneously recoded.

### SUMMARY

In Population-Hastened Assembly Genetic Engineering (PHAGE) according to the present invention, nucleic acid donors are distributed amongst a population of cells that continuously transfer nucleic acids to achieve asynchronous recoding of genetic information within a subpopulation of the cells. Recombination is achieved with biochemical systems compatible with virtually any organism.

### BRIEF DESCRIPTION OF THE DRAWINGS

Other aspects, advantages and novel features of the invention will become more apparent from the following detailed description of the invention when considered in conjunction with the accompanying drawings, all of which are incorporated by reference herein in their entirety, and wherein:

FIG. 1 shows that a directed endonuclease creates a nick offset from its recognition sequence to allow for repeated chances of Homologous Recombination (HR) with donor oligonucleotide and therefore avoiding a Non-Homologous End Joining (NHEJ) trap, according to one aspect of the invention.

FIG. 2 shows that a pair of directed endonucleases creates nicks offset from their recognition sequence, according to one aspect of the invention.

FIG. 3 shows that two pairs of directed endonucleases create nicks offset from their recognition sequence, according to one aspect of the invention.

FIG. 4 shows that a pair of directed endonucleases creates a DSB offset from their recognition sequence, according to one aspect of the invention.

FIG. 5 depicts generation of dense sequence diversity by templating a DNA break with an RNA and an error-prone reverse transcriptase (RT), according to one aspect of the invention.

FIG. 6 depicts an example biomolecular complex with both RNA-programmable recruitment of effector domains and RNA-programmable binding to DNA, according to one aspect of the invention.

FIG. 7 depicts continuous asynchronous genomic recoding with population-hastened assembly genetic engineering (PHAGE), according to one aspect of the invention.

FIG. 8 depicts searching a combinatorial library of mutations with pairwise recombinant population-hastened assembly genetic engineering (PwR-PHAGE), according to an example implementation of one aspect of the invention.

FIG. 9 depicts nanotube-assisted transport of RNA replicons, according to an example implementation of one aspect of the invention.

FIG. 10 depicts sequence specific export of RNA using RNA-binding proteins fused to an export domain and import of RNA using a self-covalent-linking pair of a ribozyme and a peptide fused to an import domain, according to an example implementation of one aspect of the invention.

FIG. 11 depicts RNA-guided programmable RNA binding with Cas9 fused to an RNA binding domain without the formation of bonded Protospacer Adjacent Motif (PAM), according to an example implementation of one aspect of the invention.

### DETAILED DESCRIPTION

In one aspect, the invention is a method for continuous genome recoding using a mixed population of cells, known as Population-Hastened Assembly Genetic Engineering (PHAGE). In PHAGE, nucleic acid donors are distributed amongst a population of cells that continuously transfer nucleic acids to achieve asynchronous recoding of genetic information within a subpopulation of the cells. Recombination is achieved with biochemical systems compatible with virtually any organism.

In a preferred embodiment, also containing a mixed population of virus, the nucleic acid content of the viruses lacks the complete set of genes necessary for viral replication and instead encodes a subset of donor oligonucleotides that template changes to the genome of interest. An infectable subpopulation of cells, referred to as “transmitters”, contain the genes necessary to allow the virus to replicate and repackage an encoding of donor oligonucleotide, again with an incomplete set of genes necessary for viral replication. Cells from another infectable subpopulation, referred to as “receivers”, do not contain the genes necessary to allow the virus to replicate and contain positions in their genome that are mutagenized by the introduction of donor-encoding oligonucleotides, plus any additional biochemical components necessary for mutagenesis. Given sufficient time, cells in the latter subpopulation will accumulate mutations from the entire set of donor oligonucleotides encoded in the genomes of the mixed viral population, while cells in the former subpopulation continue to enable viral replication.

The cell populations can be spread out as far as the viral particles can travel or be carried. For example, one embodiment may include a subpopulation of cells implanted within a multicellular organism that are “transmitters”, producing virus to infect native “receiver” cells. In order to explore combinations of alternative mutations, a given genomic position may correspond to several distinct templates encoded in the viral population. Such a relation is useful for engineering efficient gene networks. Genetic changes to “receiver” cells can modify epigenetic information, such as cytosine or histone methylation, in addition to, or instead of, nucleic acid sequences. Genetic changes also include those that do not interact with the genome, such as expression of nucleic acid constructs taken up by “receiver” cells.

One embodiment of components for efficiently stimulating mutagenesis at almost any position of the genome is a protein or RNA-directed endonuclease that nicks in the 3′ direction from its binding target recognition sequence. Since ends of a DNA break typically resect in a 5′ to 3′ direction, nicking in the 3′ direction ensures that resection will most often occur away from the recognition sequence. As a result, insertion or deletion mutations near the break that may result from non-homologous end joining (NHEJ) repair will likely occur away from the recognition sequence, which is maintained for re-targeting. Additionally, a single strand break (SSB) can induce homologous recombination with the corresponding nucleic acid donor sequence to incorporate the mutation defined by the nucleic acid template. Many specificity-programmable endonucleases producing an offset nick in the 3′ direction can work simultaneously and repeatably to mutagenize a genome of nearly all organisms.

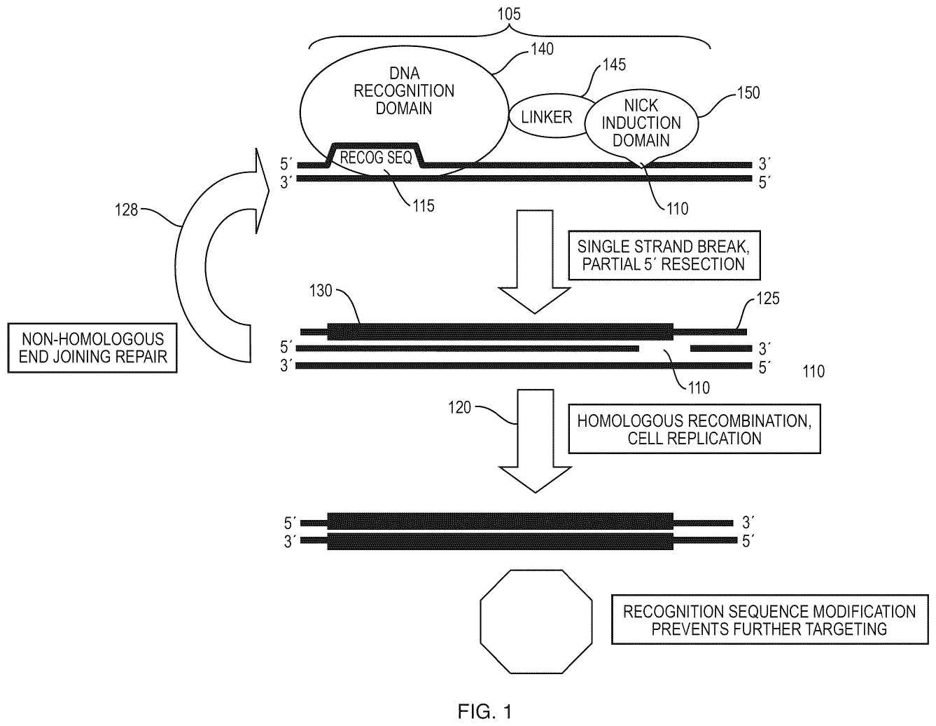

A preferred embodiment employs an engineered directed endonuclease with activity that enables scalable multiplexed genomic modifications. FIG. 1 shows that a directed endonuclease 105 creates a nick 110 offset from its recognition sequence 115 to allow for repeated chances of Homologous Recombination 120 (HR) with donor oligonucleotide 125, thereby avoiding a Non-Homologous End Joining 128 (NHEJ) trap. Thickened lines 130 indicate a region where the sequence of the template differs from the genomic DNA. As shown in FIG. 1, the activity is conferred from the structure of the engineered directed endonuclease, which consists of a DNA binding domain 140 fused 145 or interacting with a DNA endonuclease domain 150. This protein 105 is referred to as a Repeatable Directed Endonuclease (RDE).

Examples of ideal DNA binding domains for use in this aspect of the invention include Zinc Finger Nucleases (ZFNs), Transcription Activator Like Effector Nucleases (TALENs), and proteins, like Cas9, associated with Clustered Regularly Interspaced Palindromic Repeats (CRISPR) [Esvelt K M, Wang H H. Genome-scale engineering for systems and synthetic biology. Mol Syst Biol. 2013;9:641]. Examples of ideal DNA endonuclease domains include homing endonucleases (HEs) or restriction enzymes (REs) for DNA-cleaving activity. HEs (e.g. NucA, TevI, and ColE7), REs (e.g. FokI, PvuII, and MMeI), and engineered derivatives can work as monomers, heterodimers, or homodimers for cleaving on one or both strands of DNA [Beurdeley M1, Bietz F, Li J, Thomas S, Stoddard T, Juillerat A, Zhang F, Voytas D F, Duchateau P, Silva G H. Compact designer TALENs for efficient genome engineering. Nat Commun. 2013;4:1762].

The activity of an RDE can be understood by considering an example embodiment that consists of constitutive expression of dCas9 fused from its N-termini with a short flexible linker to a FokI catalytic domain (FokI-dCas9) and constitutive expression of a FokI mutant (dFokI) that does not have catalytic activity. Since dimerization is essential for FokI cleavage, a complex consisting of both FokI-dCas9 and dFokI acts as a DNA nickase. Addition of guide RNA localizes the dCas9 part of the complex to a complementary sequence of DNA and design of the linker part provides control of the nicked position and strand. Since ends of a DNA break typically resect in a 5′ to 3′ direction, nicking in the 3′ direction ensures resection will most often occur away from the recognition sequence. As a result, insertion or deletion mutations near the break that may result from non-homologous end joining (NHEJ) repair will likely occur away from the recognition sequence, which is maintained for re-targeting. Additionally, a single strand break (SSB) can induce homologous recombination (HR) with the corresponding nucleic acid donor sequence to incorporate the mutation defined by the nucleic acid template. If this mutation also eliminates part of the recognition sequence, then the mutation will be retained in the absence of further directed nicking. Creation of a SSB is less toxic to a cell than a double strand break (DSB), and more simultaneous SSB can occur simultaneously without causing unintended genomic rearrangements. Another suitable embodiment might include an engineered Cas9 with one catalytic domain deactivated, which does not have the same benefit of allowing repeatable targeting after NHEJ-related indels.

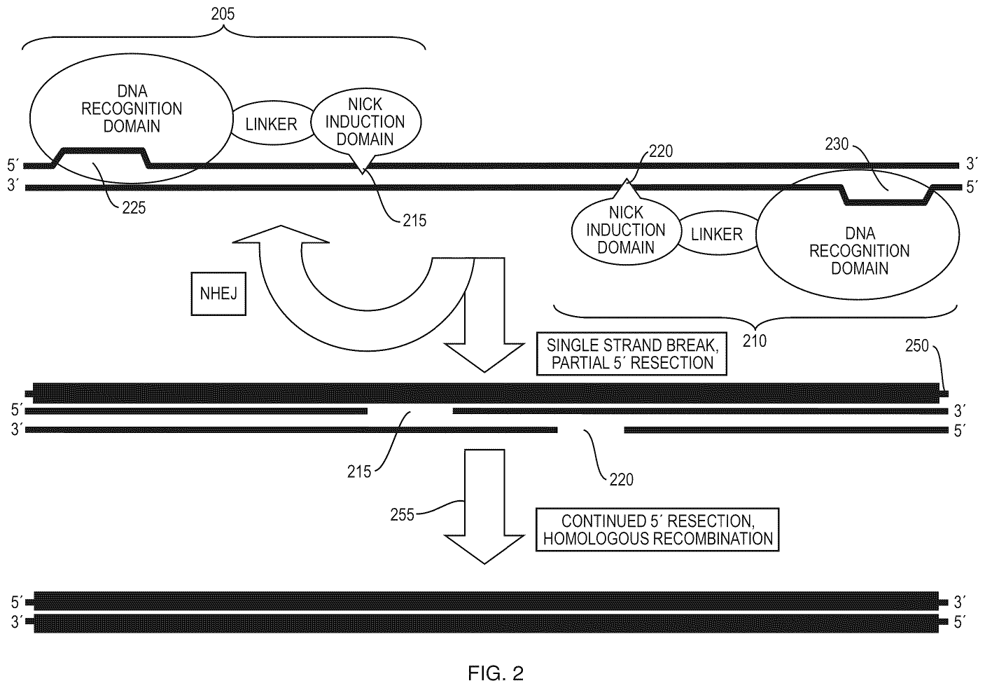

FIG. 2 illustrates the use of an RDE pair for HR that modifies both DNA strands. FIG. 2 shows that a pair of directed endonucleases 205, 210 create nicks 215, 220 offset from their recognition sequences 225, 230. They are spaced and oriented such that opposite strands are resected towards one another to eventually make a double strand break away from the recognition site of either recognition site. Again, donor oligonucleotide 250 has repeated opportunity to repair with break with Homologous Recombination 255 (HR).

Again considering the example embodiment consisting of coexpression of FokI-dCas9 and dFokI, by selecting guide RNA for recognition sequences that both orient the nick offset in the 3′ direction towards the other recognition sequence and position the two nicks within roughly 100 bases of each other [Ran F A, Hsu P D, Lin C Y, Gootenberg J S, Konermann S, Trevino A E, Scott D A, Inoue A, Matoba S, Zhang Y, Zhang F. Double nicking by RNA-guided CRISPR Cas9 for enhanced genome editing specificity. Cell. 2013 Sep. 12;154(6):1380-9], simultaneous nicks would then result in both strands 5′-resecting towards the other and ultimately a DSB. As in the case of the RDE-induced SSB, a RDE-induced DSB can induce HR with the corresponding nucleic acid donor sequence to incorporate the mutation defined by the nucleic acid template. If this mutation also eliminates part of the recognition sequence, then the mutation will be retained in the absence of further directed nicking.

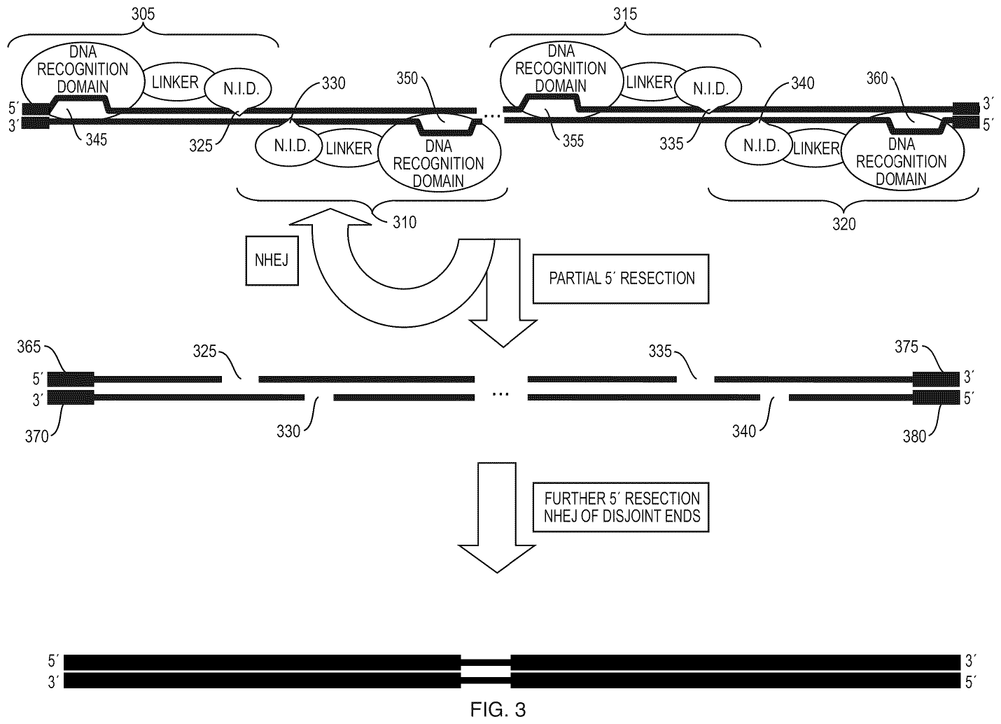

FIG. 3 shows that two pairs 305, 310, 315, 320 of directed endonucleases create nicks 325, 330, 335, 340 offset from their recognition sequences 345, 350, 355, 360. The pairs are spaced apart such that a simultaneous DSB formation between both pairs results in an excision of DNA between the pairs. Thickened lines 365, 370, 375, 380 indicate the regions flanking the exterior of all recognition sequences.

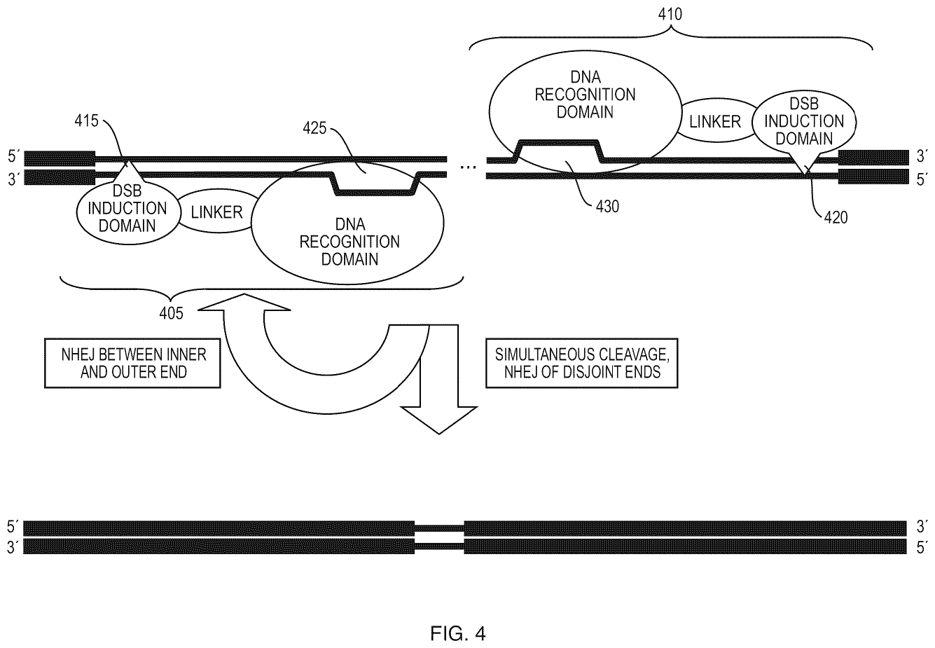

FIG. 4 shows that the same thing can be achieved with a pair of RDE that both create DSBs instead of a DNA nick. As shown in FIG. 4, a pair 405, 410 of directed endonucleases create a DSB offset 415, 420 from their recognition sequence 425, 430. A simultaneous DSB formation results in an excision of DNA between the pairs. FIG. 4 illustrates that, when using an RDE pair, a large genomic excision can be achieved even in the absence of donor nucleic acid.

A similar embodiment that primes DNA extension from nucleic acid template with either an error-prone DNA polymerase or reverse transcriptase can be used to introduce sequence diversity into genetic material. In one aspect, the invention provides an efficient method for applying in vivo transcribed nucleic acids to template repair of DNA breaks. Therefore, when the template repairs the genomic position corresponding to the template itself, mutations accumulate in the region that can be a conserved through lineage. Such an embodiment can be applied towards localized DNA sequence evolution, dynamic genome barcoding, and lineage tracing.

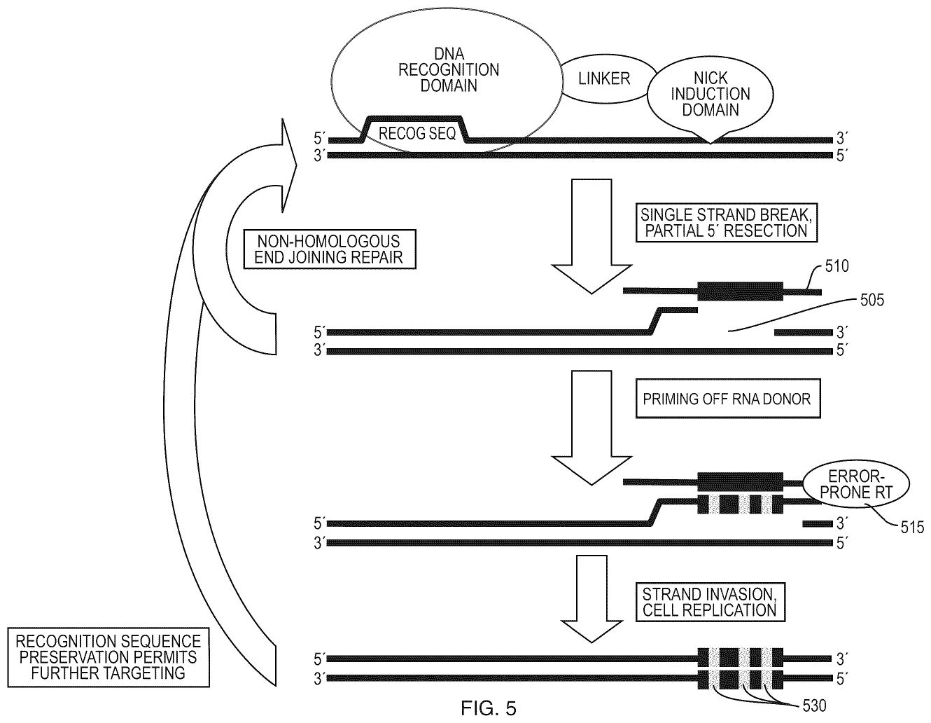

FIG. 5 depicts generation of dense sequence diversity by templating a DNA break 505 with an RNA donor 510 and an error-prone reverse transcriptase (RT) 515. Priming off the RNA donor, error-prone reverse transcription introduces random or accumulative diversity 530. Recognition sequence preservation also permits further targeting.

For some embodiments that require multiple types of genetic or epigenetic modifications, an effector corresponding each type of desired modification is linked to a unique modularly programmable RNA-binding Pumilio (Pum) [Campbell Z, Valley C, Wickens M. A protein-RNA specificity code enables targeted activation of an endogenous human transcript. Nat Struct Mol Biol. 2014 August;21(8):732-8] or Pentatricopeptide repeat (PPR) [Coquille S, Filipovska A, Chia T, Rajappa L, Lingford J P, Razif M F, Thore S, Rackham O. An artificial PPR scaffold for programmable RNA recognition. Nat Commun. 2014 Dec. 17;5:5729] protein. The recognition sites of these proteins are encoded in domains of CRISPR guide RNA that tolerate sequence-independent insertions [Silvana Konermann, Mark D. Brigham, Alexandro E. Trevino, Julia Joung, Omar O. Abudayych, Clea Barcena, Patrick D. Hsu, Naomi Habib, Jonathan S. Gootenberg, Hiroshi Nishimasu, Osamu Nureki, and Feng Zhang. Genome-scale transcriptional activation by an engineered CRISPR-Cas9 complex. Nature. 2015 Jan. 29; 517(7536):583-588]. The gRNA also directs localization of a CRISPR-associated (Cas) RNA-guided DNA-binding protein to a genomic position. The natural catalytic activity of the Cas protein is prevented by use of catalytically dead mutants, such as dCas9, or truncations to the gRNA [Kiani S, Chavez A, Tuttle M, Hall R N, Chari R, Ter-Ovanesyan D, Qian J, Pruitt B W, Beal J, Vora S, Buchthal J, Kowal E J , Ebrahimkhani M R, Collins J J, Weiss R, Church G. Cas9 gRNA engineering for genome editing, activation and repression. Nat Methods. 2015 November;12(11):1051-4].

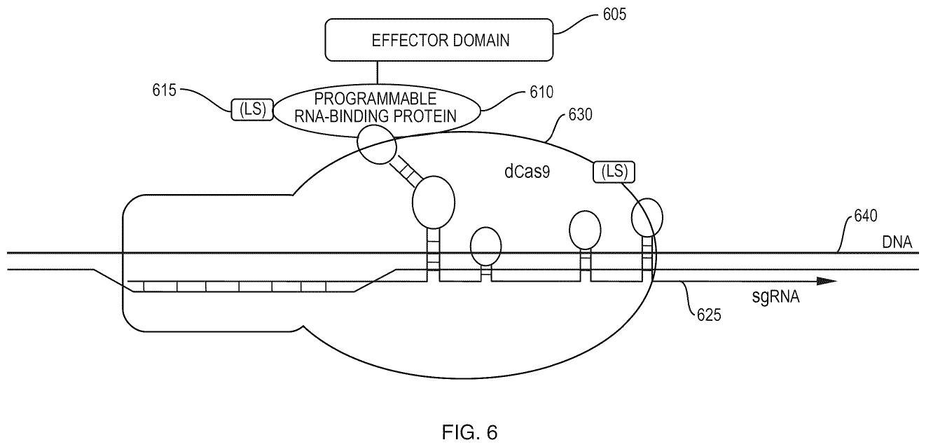

FIG. 6 depicts an example biomolecular complex with both RNA-programmable recruitment of effector domains and RNA-programmable binding to DNA. As shown in FIG. 6, effector domain 605 is linked to linked to a unique modularly programmable RNA-binding protein 610 having optional localization signal 615. gRNA 625 directs localization of a CRISPR-associated (Cas) RNA-guided DNA-binding protein 630 to a genomic position on DNA 640

An embodiment that recodes the genome exclusively with excisions consists of paired offset cleaving directed endonucleases that each target a termini of some desired excision. The endonuclease is oriented such that the target sequence is more interior than the cleavage domain with respect to the corresponding termini. Due to the repeatable activity of the endonuclease, each endonuclease continues to cleave until they simultaneously form double strand breaks (DSBs) in DNA. The fragment flanked by breakage ends is removed when NHEJ or HR ligate the other disjoint ends of the breakage. Since the fragment retains both recognition sequences, this process repeats if the fragment reinserts, repositions, or reorients.

Several embodiments of population-hastened assembly genetic engineering (PHAGE) leverage that the nucleic acid donor can either be infected [Metzger M J, McConnell-Smith A, Stoddard B L, Miller A D. Single-strand nicks induce homologous recombination with less toxicity than double-strand breaks using an AAV vector template. Nucleic Acids Res. 2011 February;39(3):926-35] or transcribed in the cell in the form of RNA or DNA [Keskin H, Shen Y, Huang F, Patel M, Yang T, Ashley K, Mazin A V, Storici F. Transcript-RNA-templated DNA recombination and repair. Nature. 2014 Nov. 20;515(7527):436-9]. Strategies for selectively producing long reverse transcribed DNA include coexpression of bacterial reverse transcriptase and retrons (e.g. those from E. coli) with synthetic insertions into their loop domain [Farzadfard F, Lu T K. Synthetic biology. Genomically encoded analog memory with precise in vivo DNA writing in living cell populations. Science. 2014 Nov. 14;346(6211):1256272] or coexpression of viral reverse transcriptase (e.g. HIV-RT) and transcripts containing at least one cognate tRNA primer binding site [Kusunoki A, Miyano-Kurosaki N, Takaku H. A novel single-stranded DNA enzyme expression system using HIV-1 reverse transcriptase. Biochem Biophys Res Commun. 2003 Feb. 7;301(2):535-9]. Alternative components may be taken from retrotransposons or group II introns [Fricker A D, Peters J E. Vulnerabilities on the lagging-strand template: opportunities for mobile elements. Annu Rev Genet. 2014;48:167-86]. Other embodiments that use RNA template can employ DNA polymerases with activity on RNA-DNA duplexes, such as Pol alpha and delta [Storici F, Bebenek K, Kunkel T A, Gordenin D A, Resnick M A. RNA-templated DNA repair. Nature. 2007 May 17;447(7142):338-41]. A reverse transcriptase from a Bordetella bacteriophage (bRT) can also template DNA polymerization from a nick with an RNA template [Doulatov S, Hodes A, Dai L, Mandhana N, Liu M, Deora R, Simons R W, Zimmerly S, Miller J F. Tropism switching in Bordetella bacteriophage defines a family of diversity-generating retroelements. Nature. 2004 Sep. 23;431(7007): 476-81]. It also contains a high adenine misincorporation rate. As previously shown in FIG. 5, error-prone polymerases like bRT can be used to generate random or accumulative diversity at programmable and precise positions on the genome.

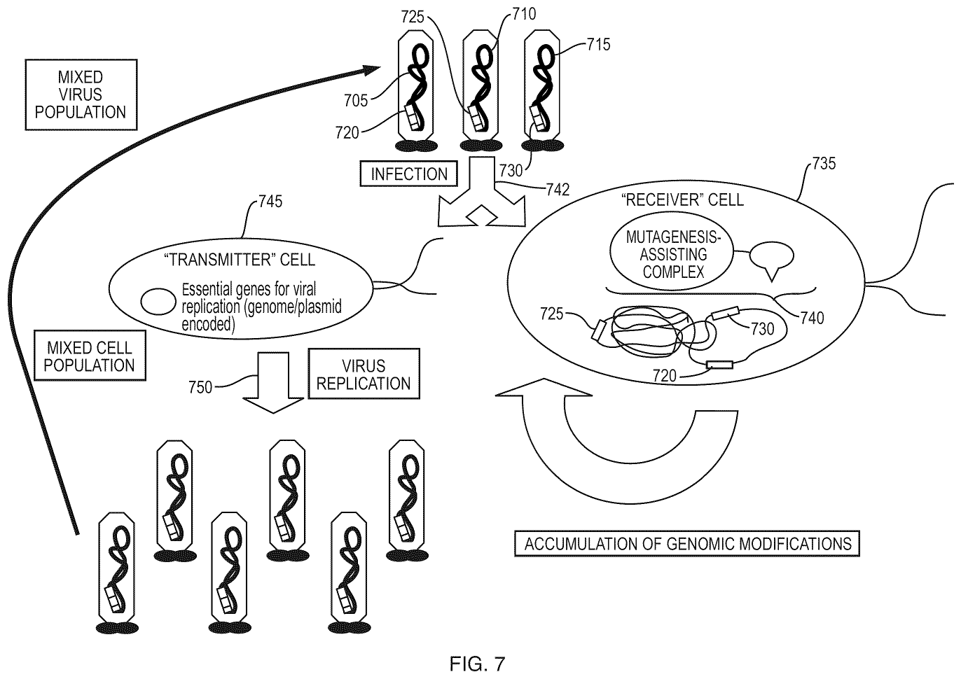

One embodiment of population-hastened assembly genetic engineering (PHAGE) according to the invention includes a mixed population of viral particles and cells. FIG. 7 depicts continuous asynchronous genomic recoding with population-hastened assembly genetic engineering (PHAGE). Viral genomes 705, 710, 715 encode a precise mutation or modification 720, 725, 730 to a position in the “receiver” cell 735 genomes. Virally-encoded guiding biomolecules direct a receiver-encoded mutagenesis-assisting complex 740 (genome/plasmid encoded), such as an offset-cutting directed endonuclease, to this position. Viruses 705, 710, 715 infect 742 both “transmitter” 745 and “receiver” 735 cells, but only replicate 750 in the former.

A potential mechanism for this selective replication can be removing genes essential for viral replication and/or packaging from the virus genome and adding them into the genetic content of the “transmitter” population. In a prokaryotic context, this can be accomplished by removing gene products 2 through 9 from M13 bacteriophage and inserting them into a plasmid in the “transmitter” population that lacks an F1 origin of replication, but contains a p15A origin of replication [ref: evo]. In a eukaryotic context, this can be accomplished by genomically encoding transfer and packaging genes, such as VSVG and Gag/Pol/Rev/Tat, in the “transmitter” cells as opposed to the viral genome. The viral genome would contain the necessary origin of replication or long terminal repeat (LTR) sites to allow its genome to be replicated and packaged in the “transmitter” population.

In many embodiments, the viral genome also expresses guiding molecules for specifying a position to mutagenize in the “receiver” population and in some cases also an oligonucleotide template for a precise mutation through processes described above. In many embodiments, the “receiver” population constitutively expresses a mutagenesis assisting biomolecule. In one embodiment, virus genomes encode retrons transcribing ssDNA and “receiver” cells express beta protein instead of or in addition to FokI-dCas9 and dFokI. In describing FIGS. 1-4, several classes of mutations were identified that are possible with the same type of RDE and can be programmable based on guide RNA. Therefore, in another embodiment, the mutagenesis assisting biomolecule can be coexpression of FokI-dCas9 and dFokI and the virus genomes expresses guide RNA and template to program a precise mutation. Other embodiments may include directed epigenetic changes with other engineered forms of Cas9 in “receiver” cells or by the virus expressing a domains with epigenetic or expression activity that can bind to an engineered RDE [Maeder et al. Targeted DNA demethylation and activation of endogenous genes using programmable TALE-TETI fusion proteins. Nat Biotechnol. 2013 December;31(12):1137-42].

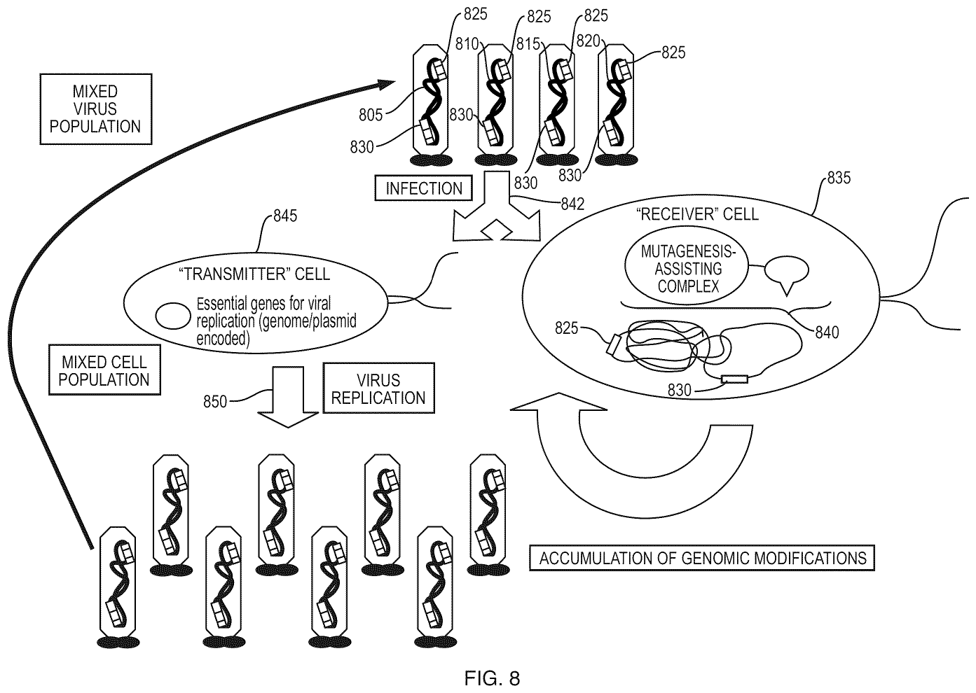

In some embodiments, introducing new sequences in the repair from one template can be used to sequence genomic modifications. Other embodiments explore a combinatorial space of changes by a viral population containing multiple potential templates for genomic positions in the “receiver” cell. An embodiment to efficiently search such a space would include pairs of template [Tsuda T. Pairwise sampling for the nonlinear interpolation of functions of very many variables. CALCOLO. 1974, Volume 11, Issue 4, pp 453-464]. FIG. 8 depicts searching a combinatorial library of mutations with pairwise recombinant population-hastened assembly genetic engineering (PwR-PHAGE. In FIG. 8, viral genomes 805, 810, 815, 820 encode two precise mutations or modifications 825, 830 to positions in the “receiver” cell 835 genomes. Virally-encoded guiding biomolecules direct a receiver-encoded mutagenesis-assisting complexes 840, such as an offset cutting directed endonuclease, to these positions. As with the system in FIG. 7, viruses 805, 810 infect 842 both “transmitter” 845 and “receiver” 835 cells, but only replicate 850 in the former.

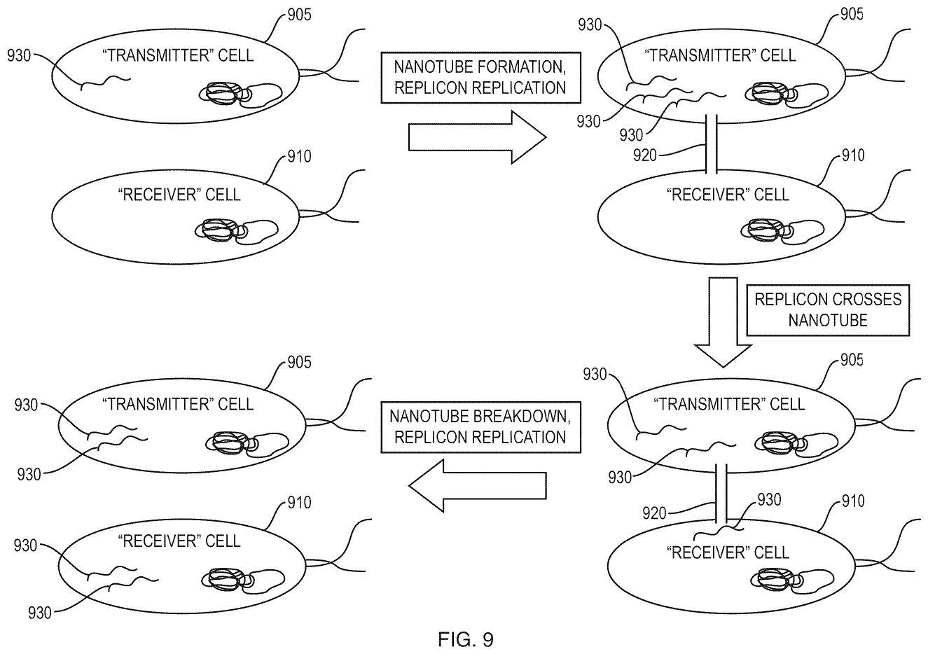

In another embodiment without the need for viral assistance, a mixed population of cells contains mechanisms for transferring nucleic acids. One such embodiment, shown in FIG. 9, relies on nanotube networks between cells that permit the transport of biomolecules [Dubey G P, Ben-Yehuda S. Intercellular nanotubes mediate bacterial communication. Cell. 2011 Feb. 18;144(4):590-600], such as self-replicating replicons [Cheng X, Gao X C, Wang J P, Yang X Y, Wang Y, Li B S, Kang F B, Li H J , Nan Y M, Sun D X. Tricistronic hepatitis C virus subgenomic replicon expressing double transgenes. World J Gastroenterol. 2014 Dec. 28;20(48):18284-95]. FIG. 9 depicts nanotube-assisted transport of RNA replicons. In FIG. 9, “transmitter” cell 905 transfers, to “receiver” cell 910 via nanotube 920, oligonucleotides 930 (replicons) that can then be translated, transcribed, and/or replicated.

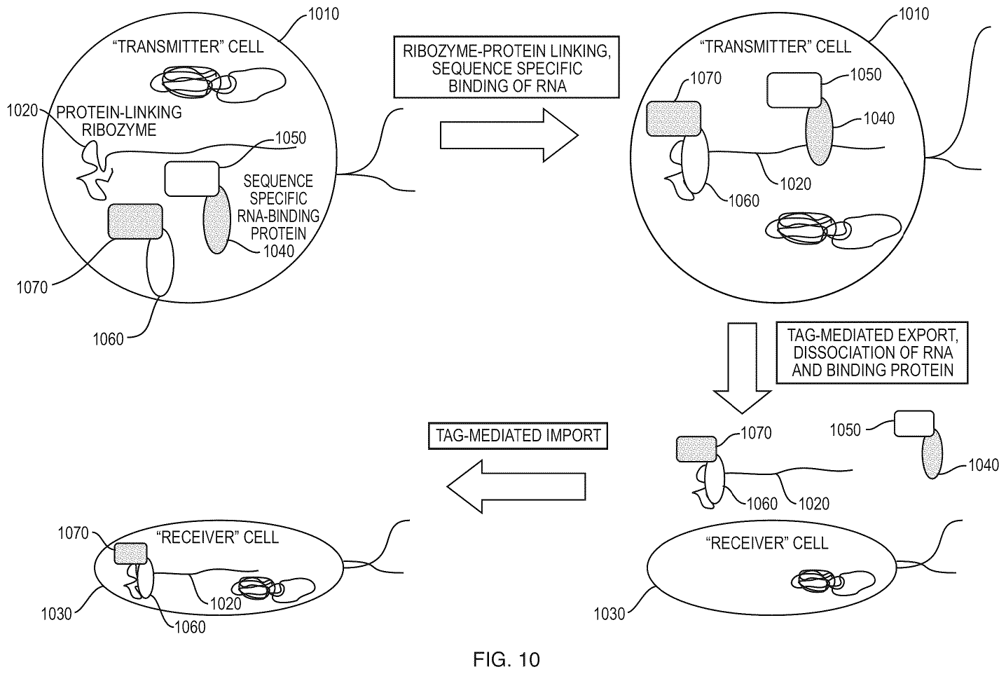

In a similar embodiment, shown in FIG. 10, “transmitter” cells 1010 selectively export nucleic acids 1020 to “receiver” cells 1030 using programmable nucleic acid binding proteins [Mackay J P, Font J, Segal D J. The prospects for designer single-stranded RNA-binding proteins. Nat Struct Mol Biol. 2011 March;18(3):256-617; Tamulaitis G, Kazlauskiene M, Manakova E, Venclovas Č, Nwokeoji A O, Dickman M J, Horvath P, Siksnys V. Programmable RNA shredding by the type III-A CRISPR-Cas system of Streptococcus thermophilus. Mol Cell. 2014 Nov. 20;56(4):506-17], protein-nucleic acid linking chemistry, protein-protein linking chemistry [Witte M D, Theile C S, Wu T, Guimaraes C P, Blom A E, Ploegh H L. Production of unnaturally linked chimeric proteins using a combination of sortase-catalyzed transpeptidation and click chemistry. Nat Protoc. 2013 September;8(9):1808-19], and/or cell export mechanisms [Lee J, Sim S J, Bott M, Um Y, Oh M K, Woo H M. Succinate production from CO₂-grown microalgal biomass as carbon source using engineered Corynebacterium glutamicum through consolidated bioprocessing. Sci Rep. 2014 Jul. 24;4:5819; Nickel W, Rabouille C. Mechanisms of regulated unconventional protein secretion. Nat Rev Mol Cell Biol. 2009 February;10(2):148-55; Regev-Rudzki N, Wilson D W, Carvalho T G, Sisquella X, Coleman B M, Rug M, Bursac D, Angrisano F, Gec M, Hill A F, Baum J, Cowman A F. Cell-cell communication between malaria-infected red blood cells via exosome-like vesicles. Cell. 2013 May 23;153(5):1120-33]. In this embodiment, “transmitter” cells 1010 also bind or encapsulate the nucleic acid 1020 with cell import [Cascales E, Buchanan S K, Duché D, Kleanthous C, Lloubès R, Postle K, Riley M, Slatin S, Cavard D. Colicin biology. Microbiol Mol Biol Rev. 2007 March;71(1):158-229] or penetration machinery [Nekhotiaeva N, Elmquist A, Rajarao G K, Hällbrink M, Langel U, Good L. Cell entry and antimicrobial properties of eukaryotic cell-penetrating peptides. FASEB J. 2004 February;18(2):394-6] for transfer into “receiver” 1030 cells. FIG. 10 depicts sequence specific export of RNA 1020 using RNA-binding proteins 1040 fused to an export domain 1050 and import of RNA using a self-covalent-linking pair 1060 of a ribozyme and a peptide fused to an import domain 1070.

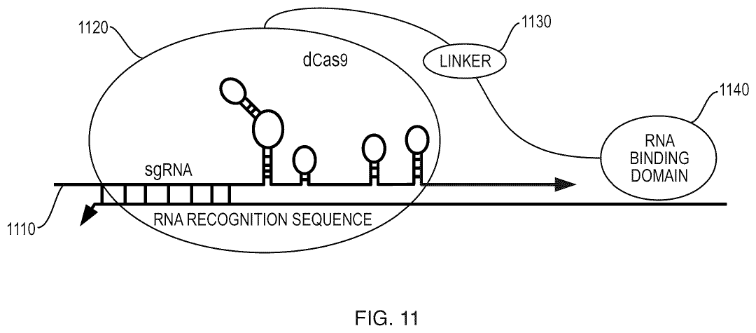

Alternatively, “receiver” cells can through import mechanisms for naked oligonucleotides. Transfer can be bidirectional to permit overlap between “transmitter” and “receiver” population. Additional localization tags can be used for greater control of the transported nucleic acid's destination. FIG. 11 depicts RNA-guided programmable RNA 1110 binding with Cas9 1120 fused 1130 to an RNA binding domain 1140 without the formation of bonded protospacer adjacent motif (PAM).

While preferred embodiments of the invention are disclosed herein and in the attached materials, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention.

What is claimed is:

1. A method for scalable multiplexed genome modification, the method comprising the steps of:
(a) providing a mixed population of cells, wherein at least some of the cells are nucleic acid donor cells that can continuously transfer donor nucleotides to other cells in the population, wherein the donor nucleotides comprise a mix of nucleotides selected to effect multiple different genome modifications, wherein at least some of the donor nucleotides encode for genome modifications at different positions of the genome to be modified than do others of the donor nucleotides, and wherein at least some others of the cells are receiver cells containing the genome to be modified and any biochemical components necessary for modification of the genome by the donor nucleotides, which components include at least one engineered directed endonuclease, wherein the cells are maintained under conditions promoting continuous transfer of donor nucleotides;
(b) simultaneously, at multiple positions of the genome to be modified, modifying the genome to be modified that is contained in the plurality of receiver cells,
wherein at least one engineered directed endonuclease in each of the plurality of receiver cells creates a break in a nucleic acid strand of the genome to be modified, wherein each engineered directed endonuclease comprises a nucleic acid recognition domain, a nucleic acid endonuclease domain, and a linker fusing or causing interaction between the nucleic acid recognition domain and the nucleic acid endonuclease domain, wherein the nucleic acid recognition domain of each engineered directed nuclease binds to a recognition sequence within the nucleic acid strand of the genome to be modified, and the nuclear acid endonuclease domain creates the break in the nucleic acid strand of the genome to be modified at a position that is outside the recognition sequence of the nucleic acid strand;
wherein homologous recombination of the nucleic acid strand with a donor nucleotide received from a donor cell occurs to create a modified genome in each of the plurality of receiver cells,
and wherein homologous recombinations are simultaneously continued in the plurality of receiver cells until all of the desired genome modifications are achieved in a majority of the receiver cells; and
(c) replicating the modified genome in the plurality of receiver cells.

2. The method of claim 1, wherein there is at least one pair of engineered directed endonucleases, and each engineered directed endonuclease of a pair creates a break in a different nucleic acid strand of a paired strand, thereby producing a modification of both strands.

3. The method of claim 2, wherein there is a plurality of pairs of engineered directed endonucleases.

4. The method of claim 1, further comprising the step of repeating steps (a)-(c) a plurality of times in order to create serial modification of the genome.

5. The method of claim 1, wherein the nucleic acid recognition domain is a DNA binding domain and the nucleic acid endonuclease domain is a DNA endonuclease domain.)

6. The method of claim 1, wherein the nucleic acid recognition domain is an RNA binding domain and the nucleic acid endonuclease domain is an RNA endonuclease domain.

7. The method of claim 1, wherein the nucleic acid recognition domain is a Zinc Finger Nuclease, Transcription Activator-Like Effector Nuclease, or a protein associated with Clustered Regularly Interspaced Palindromic Repeats.

8. The method of claim 1, wherein the nucleic acid endonuclease domain is a homing endonuclease or restriction enzyme.

9. The method of claim 1, wherein the donor cells transfer the donor nucleotides to other cells via nanotube networks between cells that permit the transport of biomolecules.

10. The method of claim 1, wherein the donor cells transfer the donor nucleotides to other cells via a mechanism comprising at least one of programmable nucleic acid binding proteins, protein-nucleic acid linking chemistry, protein-protein linking chemistry, or cell export mechanisms.

11. A method for scalable multiplexed genome modification that employs an engineered repeatable directed nuclease, the method comprising the steps of:
providing a mixed population of cells, wherein at least some of the cells are nucleic acid donor cells that can continuously transfer donor nucleotides to other cells in the population, wherein the donor nucleotides comprise a mix of nucleotides selected to effect multiple different genome modifications, wherein at least some of the donor nucleotides encode for genome modifications at different positions of the genome to be modified than do others of the donor nucleotides, and wherein at least some others of the cells are receiver cells containing the genome to be modified and any biochemical components necessary for modification of the genome by the donor nucleotides, including at least the directed endonuclease, wherein the cells are maintained under conditions promoting continuous transfer of donor nucleotides,
wherein the engineered repeatable directed nuclease comprises:
a nucleic acid recognition domain that retains the ability to bind a nucleic acid sequence at a recognition site but does not have, or has lost, an associated endonuclease activity that cleaves the nucleic acid at the recognition site;
a nucleic acid endonuclease domain; and
a linker fusing or causing interaction between the nucleic acid recognition domain and the nucleic acid endonuclease domain, wherein the nucleic acid endonuclease creates a break in a target nucleic acid strand that is offset from the recognition sequence of the nucleic acid recognition domain;
simultaneously, at multiple positions of the genome to be modified, modifying the genome to be modified that is contained in the plurality of receiver cells, wherein the nucleic acid recognition domain of the repeatable engineered directed nuclease binds to a recognition sequence within the nucleic acid strand of the genome to be modified and the break in the nucleic acid strand of the genome to be modified is outside of the recognition sequence of the nucleic acid strand, wherein homologous recombination of the nucleic acid strand with a donor nucleotide received from a donor cell occurs to create a modified genome in the receiver cell, and wherein homologous recombinations are simultaneously continued in the plurality of receiver cells all of the desired genome modifications are achieved in a majority of the receiver cells; and
replicating the modified genome in the plurality of receiver cells.

12. The method of claim 11, wherein the nucleic acid recognition domain is a DNA binding domain and the nucleic acid endonuclease domain is a DNA endonuclease domain.

13. The method of claim 11, wherein the nucleic acid recognition domain is an RNA binding domain and the nucleic acid endonuclease domain is an RNA endonuclease domain.

14. The method of claim 11, wherein the nucleic acid recognition domain is a Zinc Finger Nuclease, Transcription Activator-Like Effector Nuclease, or a protein associated with Clustered Regularly Interspaced Palindromic Repeats.

15. The method of claim 11, wherein the nucleic acid endonuclease domain is a homing endonuclease or restriction enzyme.

16. The method of claim 11, wherein the donor cells transfer the donor nucleotides to other cells via nanotube networks between cells that permit the transport of biomolecules.

17. A method for scalable multiplexed genome modification, the method comprising the steps of:
(a) providing a mixed population of viruses and cells, wherein at least some of the viruses lack a complete set of genes necessary for viral replication and instead encode for donor nucleotides, wherein the donor nucleotides comprise a mix of nucleotides selected to effect multiple different genome modifications, wherein at least some of the donor nucleotides encode for genome modifications at different positions of the genome to be modified than do others of the donor nucleotides, wherein at least some of the cells are transmitter cells that contain the genes needed for the viruses to replicate, and wherein at least some others of the cells are receiver cells containing the genome to be modified and any biochemical components necessary for modification of the genome by the donor nucleotides, which components include at least one engineered directed endonuclease, but do not contain the genes required for the viruses to replicate, wherein the mixed population is maintained under conditions promoting continuous transfer of donor nucleotides;
(b) causing at least some of the non-replicating viruses encoding the donor nucleotides to infect at least some of the transmitter cells and receiver cells;
(c) modifying the genome to be modified that is contained in the receiver cells at multiple positions of the genome simultaneously,
wherein at least one engineered directed endonuclease in each of the plurality of receiver cells creates a break in a nucleic acid strand of the genome to be modified, wherein each engineered directed endonuclease comprises a nucleic acid recognition domain, a nucleic acid endonuclease domain, and a linker fusing or causing interaction between the nucleic acid recognition domain and the nucleic acid endonuclease domain, wherein the nucleic acid recognition domain of each engineered directed nuclease binds to a recognition sequence within the nucleic acid strand of the genome to be modified, and the nucleic acid endonuclease domain creates the break in the nucleic acid strand of the genome to be modified that is outside the recognition sequence of the nucleic acid strand;
wherein homologous recombination of the nucleic acid strand with a donor nucleotide received from a transmitter cell occurs to create a modified genome in each of the receiver cells,
and wherein homologous recombinations are simultaneously continued in the receiver cells until all of the desired genome modifications are achieved in a majority of the receiver cells; and
(d) replicating the modified genome in the receiver cells.

18. The method of claim 17, wherein the viruses express guiding molecules that specify the location of the break in the nucleic acid strand to be modified in the genome of the receiver cells.

19. The method of claim 17, wherein there is at least one pair of engineered directed endonucleases, and each engineered directed endonuclease of a pair creates a break in a different nucleic acid strand of a paired strand, thereby producing a modification of both strands.

20. The method of claim 17, further comprising the step of repeating steps (a)-(d) a plurality of times in order to create serial modification of the genome.
